(19) United States
(12) Patent Application Publication
(10) Pub. No.: US 2024/0157333 A1
(43) Pub. Date: May 16, 2024

(54) PICOSCALE THIN LAYER CHROMATOGRAPHY FOR ANALYSIS OF SINGLE CELLS AND MICROSAMPLES

(71) Applicant: UNIVERSITY OF WASHINGTON, Seattle, WA (US)

(72) Inventors: Nancy Allbritton, Seattle, WA (US); Yuli Wang, Seattle, WA (US)

(73) Assignee: UNIVERSITY OF WASHINGTON, Seattle, WA (US)

(21) Appl. No.: 18/549,077

(22) PCT Filed: Mar. 7, 2022
(86) PCT No.: PCT/US2022/019103

§ 371 Date: Sep. 5, 2023

Related U.S. Application Data
(60) Provisional application No. 63/158,047, filed on Mar. 8, 2021.

(51) Int. Cl. B01J 20/283; B01J 20/10; B01J 20/28; B01J 20/30; G01N 30/93; G01N 33/92

(52) U.S. Cl. CPC B01J 20/283; B01J 20/103; B01J 20/28004; B01J 20/28035; B01J 20/28042; B01J 20/3092; G01N 30/93; G01N 33/92; B01J 2220/54; B01J 2220/82; B01J 2220/86

(57) ABSTRACT

Thin layer chromatography (TEC) devices for the analysis of pico-scale samples, methods for using the devices, and methods for fabricating the devices.

### CROSS-REFERENCE TO RELATED APPLICATION

This application claims the benefit of U.S. Application No. 63/158,047, filed Mar. 8, 2021, expressly incorporated herein by reference in its entirety.

### STATEMENT OF GOVERNMENT LICENSE RIGHTS

This invention was made with government support under Grant No. R01 CA233811 awarded by the National Institutes of Health. The government has certain rights in the invention.

### BACKGROUND OF THE INVENTION

Thin layer chromatography (TLC) is a commonly used analytical technique for separating non-volatile compounds by virtue of their differential migration in a thin layer of adsorbent matrix. Conventional TLC plates consist of an adsorbent layer (such as silica gel, aluminum oxide or cellulose) formed over a sheet of glass plate, plastic sheet or aluminum foil. TLC performance consists of three steps: spotting, development, and detection. The solution of compounds to be analyzed is first spotted onto the start line of a TLC plate. The sample solvent is allowed to evaporate, leaving behind the compounds to be separated. The TLC plate is then developed in a chamber by touching its lower edge in a solvent (eluent). The solvent is drawn into the adsorbent matrix by capillary action, moving the compounds of the samples up vertically at various rates due to their differential interactions with the matrix and solubility in the developing solvent. When the solvent front reaches a point (typically about 1 cm from the top plate edge) the plate is removed from contact with the eluent and dried. The location of the compounds on the plate can be visualized with the aid of ultraviolet (UV) light, a chemical stain, fluorescence, mass spectrometry, infrared spectroscopy, or a wide range of other methods (almost always at very low sensitivity or high signal to noise). Due to its simplicity, low cost, and speed of separation, conventional TLC is a widely used in synthetic chemistry, food, pharmaceutical, and environmental analysis.

TLC techniques have advanced significantly since the 1950s when the standardized procedure was established for conventional TLC. Conventional TLC has a typical adsorbent layer thickness of 250 μm, mean particle size of 10-12 μm and size distribution of 5-20 μm. High-performance thin layer chromatography (HPTLC) was developed in the 1970s by utilizing adsorbent particles with smaller size (5-6 μm), size distribution (4-8 μm) and layer thickness (100 μm), leading to increases in the sensitivity, resolution and speed of analysis over conventional TLC. The further improvement led to introduction of ultra-thin layer chromatography (UTLC) in 2001 by Merck. UTLC has a monolithic silica adsorbent layer with a thickness of 10 μm bound directly to the glass plates. UTLC further improves the sensitivity and decreases analysis time and separation distance compared to HPTLC. Besides monolithic silica, other materials have been used to build an adsorbent layer in UTLC including electrospun nanofibers, carbon-nanotube-templated forests, porous polymer monolithic layers, and nanostructured thin films.

In the past decades, the majority of TLC research has focused on optimization of the adsorbent layer and detection methods with aims to increase sensitivity, resolution and speed of analysis but still with a focus on large samples sizes due to the severe challenges presented by assay of ultra-small volumes. TLC technology to date has not been viewed as a method suitable for single-cell assays due to TLC's poor sensitivity, required high sample loading volumes, and modest separation capabilities. Indeed, prior attempts have demonstrated that single-cell or ultrasmall volume TLC is simply not possible despite extreme efforts and expenditure of significant resources.

Single cell analysis is a rapidly growing biomedical research field and is additionally gaining increasing prominence for clinical disease diagnosis and prognosis due to the rapid development of highly sensitive, integrated technologies such as micro/nanofluidic devices. The heterogeneity of cells within a population is now well recognized as both a disease driver as well as fundamentally important in the understand of nearly all aspects normal physiology. Further these micro/nanofluidic and array-based devices are readily scaled to match the dimensions and volumes of a single cell to achieve assay success and appropriate detection signal to noise. As mentioned above one of major obstacles for using TLC for single cell analysis is that the dimension of TLC adsorbent matrix (thickness: 10 to >100 μm, width: >1 cm) significantly exceeds that of single cells (typical diameter and thickness: 10-20 μm, volume: 1-2 pL). As a result, the compounds eluted from a single cell are diluted laterally and vertically in the matrix quickly reaching concentrations below the limit of detection. To address this challenge, the thickness and width of the porous matrix of TLC needs to be miniaturized into microbands with a thickness/width comparable to the size of single cells. This miniaturization is expected to reduce the lateral diffusion and confine the movement of compounds along the microbands. The sample loading zone and workflow must also be appropriate to that of a single cell. Although a few previous publications have created TLC bands as separation lanes, their band width exceeds 200 μm and none of them has miniaturized the TLC bands with aim for single cell analysis. Additionally, the lane thickness (≥200 μm) is far too great to support small-sized sample assays. These methods while interesting demonstrate no benefit over standard TLC with respect to ultra-small sample assay.

A wide range of detection strategies have been combined with TLC, including absorbance, fluorescence, colorimetric development, mass spectrometry, and various spectroscopy strategies. Despite intensive effort, detection limits remain relatively poor compared to other separation methods with limit of detection (LOD) typically of 10⁻¹⁴-10⁻¹⁰ moles/spot. This is due to sample dilution, matrix noise, low or zero off rate analyte adsorption. Efforts to optimize or eliminate these undesirable matrix challenges have not been successful. Sample deposition typically utilizes glass capillary tubes to spot liquid volumes of 0.1-0.5 μL (for HPTLC) with poor spatial precision (i.e., typically at 1 spot per 1 cm wide plate). Thus, conventional TLC, HPTLC and UTLC are not able to achieve the demanding specifications required for analysis of ultra-small volumes.

A need exists for improved TLC devices capable of analysis of ultra-small volumes and methods for fabricating TLC devices having improved performance, particularly analysis of ultra-small volumes. The present invention seeks to fulfill this need and provides further related advantages.

### SUMMARY OF THE INVENTION

The present invention provides a chromatography device (thin layer chromatography (TLC) device), methods for fabricating the device, and methods for using the device.

In one aspect, the invention provides a thin layer chromatographic (TLC) device, comprising a non-porous substrate having one or more microchannels formed on its surface, each microchannel comprising a porous, substantially homogeneous microband adapted for fluid flow driven by capillary action.

In another aspect, method for fabricating a thin layer chromatography device, comprising:
(a) forming one or more microchannels in a non-porous substrate;
(b) filling the one or more microchannels with a flowable porous microband precursor composition; and
(c) subjecting the non-porous substrate having one or more microchannels filled with a flowable porous microband precursor composition to conditions to that convert the composition to a porous microband to provide a thin layer chromatography device.

In a further aspect, the invention provides methods for thin layer chromatography of the contents of a single cell, a group of cells, or a microsample using the chromatography device described herein.

### DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIG. 1 illustrates the concept of separating compounds from small scale samples such as single cells or other small-scale samples by picoscale thin layer chromatography (pTLC). Panel A illustrates a representative pTLC plate that includes porous microbands (single microband) formed on a non-porous substrate (e.g., glass slide). Panel B illustrates compounds in cells that are stained with markers (e.g., fluorophores), and a single cell or other sample is dispensed at the left side of the microband. Panel C illustrates applying solvent at the left end of the microband and its migration to the right end. The stained compounds in the cell or other deposited sample are extracted by the solvent. The compounds are separated due to their differential migration speed and interactions with the matrix.

FIGS. 2A-2C illustrate the fabrication of pTLC plate. FIG. 2A is a schematic illustration of a representative fabrication process (PDMS: polydimethylsiloxane. TMOS: tetramethyl orthosilicate. PEG: polyethylene glycol). FIG. 2B shows bright field microscopic images of the pTLC plate (Top: low magnification top view. Middle: high magnification top view. Bottom: high magnification side view). FIG. 2C is an illustration demonstrating the porous structure of silica microbands. Left two bands are dry and dark. Right two bands are immersed in water and become transparent.

FIGS. 3A-3C illustrate the separation of spotted lipid mixtures by pTLC. FIG. 3A is a schematic illustration of a setup for development and imaging. FIG. 3B shows the separation of 18:1 PE CF and Texas Red DHPE by 1-butanol:water:acetic acid (18:4:4 vol:vol:vol). FIG. 3C shows the chemical structures and of separation of sphingosine fluorescein (SpF) and sphingosine-1-phosphate fluorescein (S1PF) by 1-butanol:water:acetic acid (18:1:1 vol:vol:vol).

FIGS. 4A-4C illustrate the separation of lipophilic red/green lipids from single cells by pTLC. FIG. 4A show the chemical structure of DiO and DiD. FIG. 4B shows U937 cells stained with DiO and DiD. FIG. 4C are time lapse images showing the separation of DiO and DiD from a single U937 cell by 1-pentanol.

FIGS. 5A-5D illustrate the separation of sphingosine metabolites from single cells by pTLC. FIG. 5A illustrates a schematic of the process. Sphingosine (Sp) alkyne is loaded into U937 cells. Inside the cells, sphingosine alkyne is converted to sphingosine-1-phosphate (S1P) alkyne by sphingosine kinase. The cells are then fixed, and the alkyne moieties are reacted with azide-containing fluorophore (e.g., CY5-azide) through click chemistry (chemical structures of Sp alkyne, S1P alkyne and CY5-azide are shown). FIG. 5B illustrates U937 cells loaded with Sp alkyne and clicked with CY5-azide. FIG. 5C are time lapse images (0, 2, 4, 6, and 11 minutes) showing the separation of Sp-CY5 and S1P-CY5 from a single U937 cell. The developing solution is 1-butanol:1-propanol:water:triethylamine (18:2:4:1, vol:vol:vol:vol). FIG. 5D is a TLC chromatogram at 11 min showing Sp-CY5 is completely separated from S1P-CY5.

### DETAILED DESCRIPTION OF THE INVENTION

The present invention provides a chromatography device (thin layer chromatography (TLC) device), methods for fabricating the device, and methods for using the device.

Thin layer chromatography (TLC) is a widely used analytical technique for a variety of applications, but its use for extremely miniature specimens, such as a single cell with the volume as small as a magnitude of 1 picoliter, has not been realized so far. A major challenge is that the dimensions of adsorbent layer in conventional TLC significantly exceeds that of single cells. The large volume of the adsorbent does not limit diffusional spread of the ultra-small volume resulting in sample dilution during the chromatographic separation step. Detection of such microsamples is a major limitation due to the very small number of sample molecules (10⁻²¹ to 10⁻¹⁵ moles) as well as background noise created by the adsorbent (light scattering and light absorption by the matrix, for example). Sample nonspecific binding to the adsorbent with a zero or very slow off rate (or range of off rates) create further difficulties that must be overcome for assay of these limited samples. Other challenges include the method use to deposit the sample in a small volume and precise location—neither is of sufficient volumetric or spatial resolution in conventional TLC for ultrasmall samples. Dispersion due to large sample volumes (required by conventional TLC) or sample spreading due to surface tension forces must be overcome as well as the ability to place the sample with high spatial resolution (≤50 μm) at the entry to the sample region for success in assay of ultrasmall volumes. Conventional TLC is unable to meet these demanding specifications.

To address this challenge, the present invention provides a TLC platform (termed picoscale Thin Layer Chromatography (pTLC)) that combines sol-gel chemistry and microfabrication technology. pTLC consists of a single or an array of microscale bands made from highly porous monolithic silica (or other material) designed to accept picoliter-scale volume samples. In certain aspects, the present invention combines highly sensitive detection methods as well as separation optimization to overcome the additional limitations of TLC methodologies to date. In certain embodiments, the invention provides a TLC device in which the width and depth of each band is comparable to the size of single cells (≤100 μm). As described below, to demonstrate proof-of-principle of ultra-small sample analysis, model fluorescent compounds (18:1 PE CF and Texas Red DHPE, sphingosine fluorescein and sphingosine 1-phosphate fluorescein) were micro-spotted on the microband and successfully separated by pTLC. As further described below, to demonstrate its capability to separate picoliter volumes, a mammalian single cell loaded with fluorescent compounds (DiO and DiD, CY5 labeled sphingosine metabolites) was spotted on a microband, and the compounds were extracted from single cells and separated by pTLC. Thus, in certain aspects, the present invention provides a device and method for single cell analysis and femto- to nanoliter-scale specimens.

The devices and methods described herein are useful in applications where miniature specimens and high throughput parallel analyses are needed, such as diagnosis of diseases, cell heterogeneity analysis, monitoring droplet-based reactions, detecting miniature environmental, food, forensic and pharmaceutical samples. A powerful asset of the devices and methods described herein lies in their simplicity and high-speed separation capabilities. For example, the devices described herein are readily operated without active fluidic controls (pumps, valves, flow meters), current flows, or fluid-level balancing. By a simple evaporation of solvents, separation can be terminated for post-chromatographic detection. These features make the device robust yet simple to operate enabling fully enclosed cassette (e.g., at home assays as well as highly scalable for automated high throughput operations, such as drug discovery or combinatorial chemical reactions).

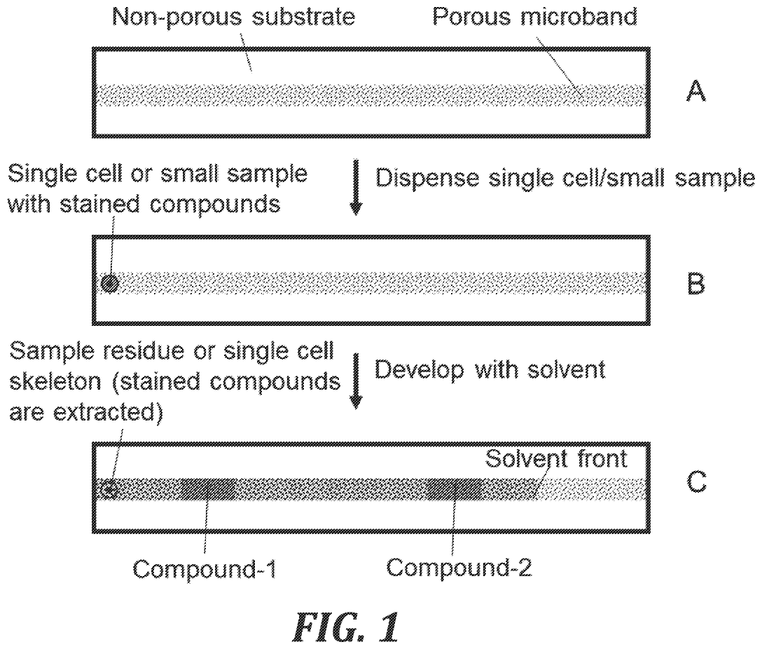

The present invention provides a TLC platform for analyzing extremely miniature samples and specimens, such as a single cell. The platform is suitable for assay of a wide range of ultrasmall volume samples (1 zeptoL (zL), 1 attoL (aL), 1 femtoL (fL), 10 fL, 100 fL, 1 pL, 10 pL, 100 pL, 1 nL, 10 nL, 100 nL). Molar sample size ranges include 10⁻²¹, 10⁻²⁰, 10⁻¹⁹, 10⁻¹⁸, 10⁻¹⁷, 10⁻¹⁶, and 10⁻¹⁵ moles. The concept of using pTLC for separating compounds from small sample volumes such as a single cell is illustrated in FIG. 1. Besides cells, this concept can also be applied to other miniature samples in the forms of droplets, aerosols, crystals, fibers, microcapsules, microbeads, microgels, liquids, solids, gels, oils, pico to nano-liter containers, microplastics, microparticles, prions, aggregates, liposomes and other membrane-bound vesicles/structures, plaques, films, precipitates, and particles. The pTLC plate consists of an array of microscale bands made from a porous matrix on a non-porous substrate (FIG. 1A, only one band is shown). The dimensions of a band are comparable to the size of single cell size (typical diameter: 10-20 μm but also smaller and larger cells and organisms such as viruses, bacteria, fungi, yeast, microorganisms, plankton, algae, mammalian and plant cell, oocytes, sperm, nematodes, insects, scales, hair). Small sample sizes can include collections of cells such as organoids, cell colonies, biopsies, aspirates and other biological samples such as plasma, blood, urine, membranes, lymph, cerebral or spinal fluid, tears, saliva, bile, feces, sweat and other excretions/secretions. Compounds within cells, outside of cells or secreted by cells might be samples. Subcellular organelles are suitable samples such the nucleus, Golgi apparatus, mitochondria, endoplasmic reticulum, secretory or proteolytic or synaptic vesicles, lysosomes, vacuoles, extracellular vesicles, phagosomes, nucleolus, ribosome, cytosol, or centrosome. The microscale bands have a width ranging 1-1000 μm, a depth ranging from 0.5-100 μm, and a length ranging 0.1-100 mm. Thus, sample dimensions or spot sizes can be in the range of 50 nm, 100 nm, 1 micron, 10 microns, 1000 microns). The compounds found in the small sample may or may not be stained with markers (e.g., fluorophores, chemical stains, antibodies, rare metal isotopes, radioactive isotopes, click reagents, cross-linkers, nanoparticles, quantum dots, p-dots, lanthanides), either before or after the separation step and a single cell or other small-volume sample is dispensed at the left side of the microband (FIG. 1B). Solvent is applied at the left end of the microband and its migration is drawn along the microband in the direction of the capillary force. The stained compounds are eluted from the cells or sample spot, and their migration is confined along the band and separated downstream due to their differential migration speed (FIG. 1C).

### TLC Devices

In one aspect, the invention provides chromatographic devices. In certain embodiments, the invention provides a thin layer chromatographic (TLC) device, comprising a non-porous substrate having one or more microchannels formed on its surface, each microchannel comprising a porous, substantially homogeneous microband adapted for fluid flow driven by capillary action.

In certain embodiments, the fluid flow is capillary fluid flow combined with electrophoresis. In other embodiments, the fluid flow is capillary fluid flow driven by intermolecular forces. In the device, the fluid flow is not driven by hydrodynamic centrifugal force or pressure-driven fluid flow. Solvent can be drawn into the porous microband and then migrated along the band by capillary force, pressure, vacuum, centrifugation force, electrophoretic force, osmotic force, or magnetic force, among others. Solvent combinations can be used and applied to microbands in either discontinuously or in gradients.

As used herein, the term “microchannel” refers to a channel that is formed in a surface of the non-porous substrate. During TLC device fabrication as described herein, each microchannel receives the flowable composition (e.g., silica sol-gel slurry) that is subsequently processed in situ to provide the microband. The microchannel can have a variety of shapes. For example, the microchannel can be U-shaped, V-shaped, include three (3) sides of a rectangle or square, or include the bottom portion of a triangle/pentagon/hexagon. In certain embodiments, the microchannel has a flattened U-shape. In other embodiments, the microchannel includes a first surface (bottom) that is substantially parallel to the surface of the substrate and second and third opposing surfaces (sides) that are substantially perpendicular to the first surface (i.e., a three-sided channel).

The term “porous microband” refers to a porous matrix suitable for chromatography. Each porous microband is formed in situ in the microchannel. The microband is substantially homogeneous (e.g., crack-free, fracture-free, defect-free) porous matrix through which samples migrate. In one embodiment, the microband is a porous crack-free silica monolithic matrix having a width from about 1 to about 1000 μm, a depth from 1 to 100 μm, and a length from about 0.1 to about 100 mm.

The microband is porous matrix having a pore size from nanometers to micrometers (e.g., from about 1 nm to about 999 μm). The term “porous” refers to the microband that fills the microchannel and through which the sample migrates in the TLC device. Suitable porous microbands include inorganic (e.g., silicon, silicon oxide, silica gel, aluminum oxide, titanium oxide, carbon nanostructures), metal (e.g., gold, silver, titanium), organic (e.g., polymeric such as polymer nanofibers, polymer hydrogels, polyamides, monolithic porous polymers), natural (e.g., chitosan, diatomaceous earth, diatom biosilica, cellulose), plastic, and hybrid materials. Representative materials for the porous microbands include diatomaceous earth, cellulose (and modified celluloses), polyamide, modified/coated silicas (amino-bonded silica, cyano-bonded silica, thiol-bonded silica, diol-bonded silica, C-18, C-8, and C-2 bound silicas, phenyl-silica and others), hydroxyl apatite, zinc carbonate, polyethylene, calcium sulfate, calcium hydroxide, dextran, polyacrylonitrile, polycaprolactone, calcium hydroxide, magnesium silicate, magnesium oxide, starch, agar, alumina, titania, indium tin oxide, and combinations of above (such as silica-titania). Representative porous microbands include silica-based microbands.

In certain embodiments, the porous microband is monolithic porous silica.

In one embodiment, the thin layer chromatographic (TLC) device comprises a non-porous substrate having one or more microchannels formed on its surface, each microchannel comprising a monolithic porous silica microband adapted for fluid flow driven by capillary action.

It will be appreciated that although the TLC devices described herein have been demonstrated to be advantageous for monolithic porous silica microbands, other porous matrices made from materials other than silica are useful for the microbands of the TLC devices described herein and are within the scope of the invention.

In certain embodiments, the porous microband has a width from about 1 to about 1000 μm (e.g., ≤5, ≤15, ≤30, ≤50, ≤100, ≤200, ≤500, or ≤1000 μm). In certain of these embodiments, the porous microband has a width from about 50 to about 150 μm. In other embodiments, the porous microband has a width from about 50 to about 100 μm. In further embodiments, the porous microband has a width from about 50 to about 75 μm. In certain embodiments, the porous microband has a width less than 200 μm (e.g., less than 190 μm). In representative embodiments, the porous microband has a width of about 1 μm, about 5 μm, about 10 μm, about 50 μm, about 100 μm, about 200 μm, about 500 μm, or about 1000 μm. The porous microbands of the TLC devices described herein have the widths described and are discontinuity-free (e.g., crack-free, fracture-free, defect-free).

In certain embodiments, the porous microband has a depth from about 1 to about 100 μm (e.g., ≤5, ≤15, ≤30, ≤50, or ≤100 μm). In certain of these embodiments, the porous microband has a depth from about 10 to about 50 μm. In other embodiments, the porous microband has a depth from about 15 to about 25 μm. In further embodiments, the porous microband has a depth of about 15 μm.

In certain embodiments, the porous microband has a length substantially co-terminous with the length of the device. Representative lengths are from about 0.1 to about 100 mm.

In certain embodiments, the porous microband has an aspect ratio (width/depth) from about 1 to about 100. Representative useful aspect ratios are 0.1, 0.5, 1, 5, 10, 5, 10, 50, and 100.

In certain embodiments, the porous microband has a cross-sectional area from about 10 μm² to about 50,000 μm². Representative useful cross-sectional areas are 10 μm², 100 μm², 500 μm², 1,000 μm², 5,000 μm², 10,000 μm², and 50,000 μm².

In the TLC device, each microchannel comprises a porous, substantially homogeneous microband. This is achieved by the fabrication process in which flowable precursors of porous material fill each microchannel and in the process take the shape of the microchannel thereby filling the channel. The solid, porous microband occupying each microchannel is formed from the precursors by further processing as described herein. The solid, porous microband so formed is also homogeneous by virtue of the use of flowable sol-gel precursors to fill each microchannel. As used herein, the term “homogeneous” refers to the porous microband's composition, mechanical properties (e.g., strength, stability, and continuous, crack-, fracture-, defect-, discontinuity-free), and porosity properties (e.g., pore size (1 nm to <10 μm), pore size distribution, and distribution of pores throughout the microband).

In certain embodiments, the microband is a patterned microband having zones or regions with different properties. In certain of these embodiments, the microband includes regions that are crosslinkable effective to trap analyte bands.

In certain embodiments, the microchannel and ultimately the microband is straight or curved (e.g., zig-zag, spiral, circular).

In certain embodiments, the microchannel and ultimately the microband is in the non-porous substrate (e.g., via photolithography or microfabrication processes). In other embodiments, the microchannel is formed on top of the non-porous substrate (e.g., via micromolding in capillaries) followed by cladding laterally with non-porous walls on two sides of the microband.

The term “non-porous” refers to a substrate material that is non-porous to solvents and solvent systems used to elute samples in the TLC device. Suitable non-porous substrates include inorganic (e.g., glass, silicon, quartz), metal, plastic, or organic (e.g., polymeric) substrates. The non-porous substrate can be inorganic (e.g., glass, silicon, quartz), organic (e.g., polymer plate), or metal. In certain embodiments, the substrate is glass, quartz, plastic, polymer, ceramic, metal, or silicon. In certain embodiments, the substrate is glass.

In certain embodiments, the device comprises a single microchannel comprising the porous, substantially homogeneous microband.

In other embodiments, the device comprises from two to about 1,000,000 microchannels, each microchannel comprising the porous, substantially homogeneous microband. A typical TLC plate is 20 cm×20 cm. For such a plate, in one embodiment, the device includes 10,000 channels (10 μm width, 10 μm gap). In another embodiment, the device includes up to 2,000 channels (50 μm width, 50 μm gap). In a further embodiment, using higher precision fabrication techniques, (e.g., laser machining, e-beam), the device includes up to 100,000 channels (10 μm width, 5 μm gap).

Representative TLC devices of the invention differ from other conventional TLC devices. Conventional TLC, HPTLC, UTLC are continuous and do not rely on or use microbands. Narrow bands of conventional TLC devices are not the microbands of the TLC devices described herein. Although narrow bands are described in conventional devices these narrow bands have widths that exceed 200 μm and none have dimensions of the microbands of the representative TLC devices of the invention as described herein.

Conventional TLCs are defined as “continuous” because their porous stationary phase is not cladded laterally with nonporous wall. The microbands of the devices described herein are not “continuous” because they are cladded laterally with non-porous walls. As a result of this cladding, the movement of analytes is restricted along the microbands without lateral diffusion.

The TLC devices described herein can be utilized to
chromatograph the contents of a single cell;
chromatograph the contents of a single organism;
chromatograph the contents of a viral particle;
chromatograph analytes in samples having a volume from about 1 zL to about 100 nL;
chromatograph samples in forensic drug analysis, toxicology, explosives analysis, and analysis of dyes and inks for the identification and comparison of drugs, explosives, inks and dyes, toxins, and pesticides; and
assay drug candidates and drug metabolites.

### Methods for TLC Device Fabrication

In another aspect, methods for fabricating chromatography devices are provided. In certain embodiments, the invention provides a method for fabricating a thin layer chromatography device, comprising:
(a) forming one or more microchannels in a non-porous substrate;
(b) filling the one or more microchannels with a flowable porous microband precursor composition; and
(c) subjecting the non-porous substrate having one or more microchannels filled with a flowable porous microband precursor composition to conditions to that convert the composition to a porous microband to provide a thin layer chromatography device.

In certain embodiments, the fabrication includes the following steps:
(1) form microchannels in a glass;
(2) temporarily seal the microchannels with an elastomer slab (such as PDMS);
(3) fill the microchannels with flowable silica sol-gel slurry;
(4) convert the flowable sol-gel composition to a crack-free, monolithic silica porous structure (gelling and aging); and
(5) reveal the porous microbands by detaching elastomer slab from glass.

The fabrication method advantageously includes step (2): sealing the microchannel with an elastomer slab. This step provides the enclosed microchannels that are required to carry out step (3) and (4).

In certain embodiments of the TLC device described herein, the porous microband is a porous matrix made of monolithic porous silica. Monolithic porous silica is difficult to microfabricate into microbands due to crack formation. Traditionally, monolithic porous silica is formed inside a column or capillary by sol-gel chemistry. To date, fabrication of monolithic porous silica microbands as described herein is unknown. The fabrication method described herein, for example, the combination of steps (1)-(5) above, provides a novel crack-free (fracture-free, and defect-free) monolithic porous silica microband (cladded with non-porous wall).

It will be appreciated that although the fabrication method has been demonstrated to be advantageous for monolithic porous silica, other porous matrices comprising materials other than silica are useful for the microbands of the TLC devices described herein and are within the scope of the invention.

As noted above, in certain embodiments, the method comprises enclosing the one or more microchannels prior to filling (e.g., PDMS slab) with the precursor composition. In certain embodiments, the precursor composition comprises tetramethyl orthosilicate, a polyethylene glycol, and urea.

In certain embodiments, subjecting the non-porous substrate to conditions to that convert the precursor composition to a porous microband comprises polymerizing the precursor composition to provide a gel, subjecting the gel to heat (120° C.) and pressure (15 PSI) to provide a porous gel (Ostwald ripening), exposing the porous gel by removing the enclosure, and then subjecting the non-porous substrate with exposed porous gels to heat (330° C.) to calcinate the porous gel to provide non-porous substrate comprising one or more monolithic porous silica microbands.

The microband shape, width, depth, length, aspect ratio, cross-sectional area, and composition is as described above for the TLC device.

The non-porous substrate is as described above for the TLC device.

In certain embodiments, the microchannel is in the non-porous substrate surface (e.g., via photolithography or microfabrication processes) or formed on top of the non-porous substrate (e.g., via micromolding in capillaries) followed by cladding laterally with non-porous walls.

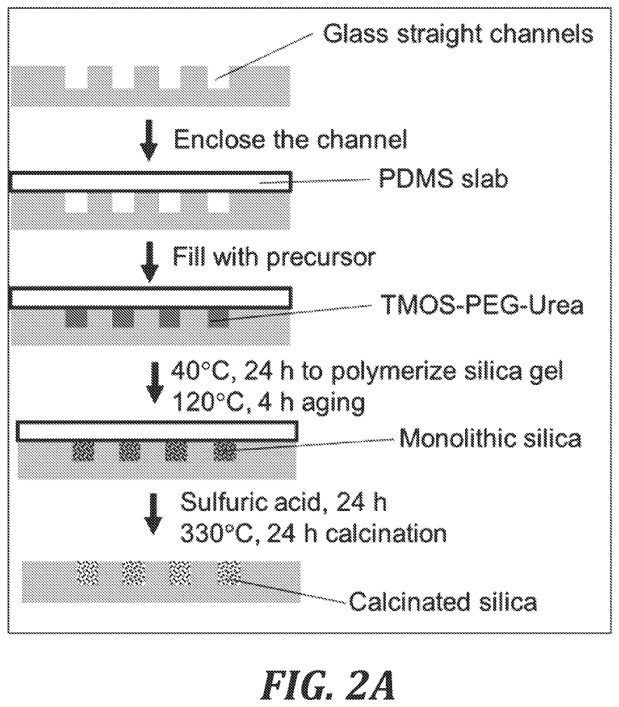

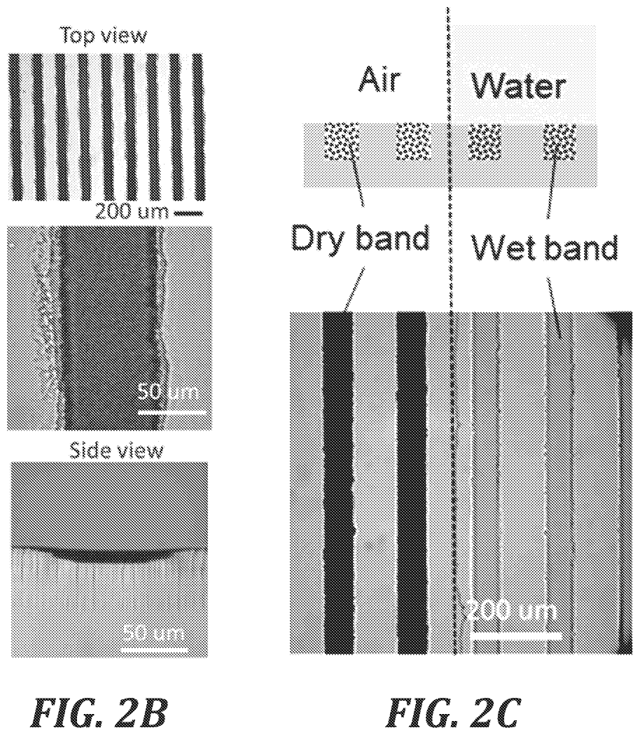

A schematic of a representative fabrication process is shown in FIG. 2A. The process illustrates sol-gel micromolding in microcapillaries (i.e., microchannels). Referring to FIG. 2A, a soda lime glass slide (3 inches×1 inch) possessing an array of open, straight microfluidic channels was fabricated by photolithography and wet etching (see, e.g., Castano-Alvarez, M.; Ayuso, D. F. P.; Granda, M. G.; Fernandez-Abedul, M. T.; Garcia, J. R.; Costa-Garcia, A., Critical points in the fabrication of microfluidic devices on glass substrates. Sens. Actuator B-Chem. 2008, 130 (1), 436-448). The channels in this example have a width of 60-80 μm, a depth of 13 μm and a length of 60 mm. To enhance the bonding of glass with silica monolith, the channel surface was roughened by etching with 7.5% sodium bicarbonate aqueous solution at 70° C. for 24 h. The channels were enclosed by attaching a polydimethylsiloxane (PDMS) slab. Monolithic silica sol-gel precursor was prepared by mixing 5.6 mL tetramethyl orthosilicate (TMOS), 1.2-1.5 g polyethylene glycol (PEG, MW=10,000 g/mole), 0.9 g urea, and 10 mL aqueous acetic acid solution (10 mM) on ice for 45 min. The channels were then filled with the precursor and incubated in a humidified chamber at 40° C. for 24 h to polymerize the silica gel. PEG acted as a phase separation inducer to form the macro-porous silica structure. After gelation, the glass slide (with attached PDMS slab) was immersed in 45 mL of 10 mM aqueous acetic acid solution containing urea (yielding a concentration of 0.09 g/mL urea) in a polypropylene Coplin jar and autoclaved at 120° C. at 15 pounds per square inch (PSI) for 4 h to form mesopores according to the Ostwald ripening process. The plate was then rinsed with water and dried in air. The PDMS slab was then detached from the glass slide to expose the silica microbands. The slide was then incubated in water for 2 h to leach PEG and urea out from the silica monolith. To further remove the organic polymer PEG from the silica monolith, the slide was immersed in sulfuric acid for 24 h. The slide was then incubated in water for 24 h to remove the sulfuric acid. Finally, the monolith/slide was calcinated by baking on a hotplate at 330° C. for 24 h to provide a mechanically stable silica monolith.

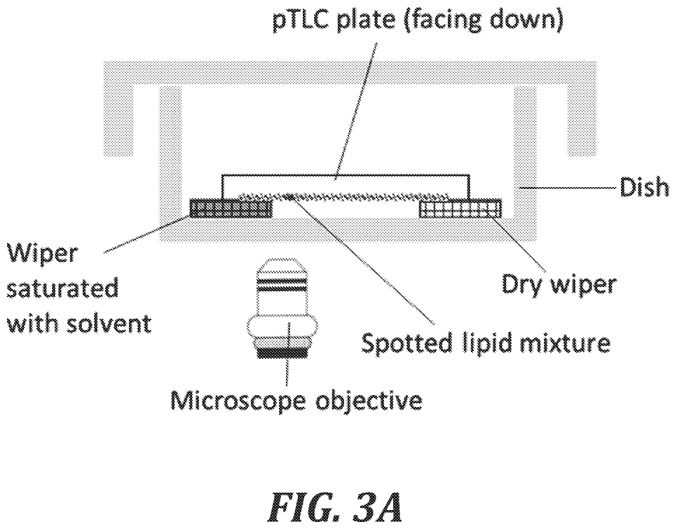

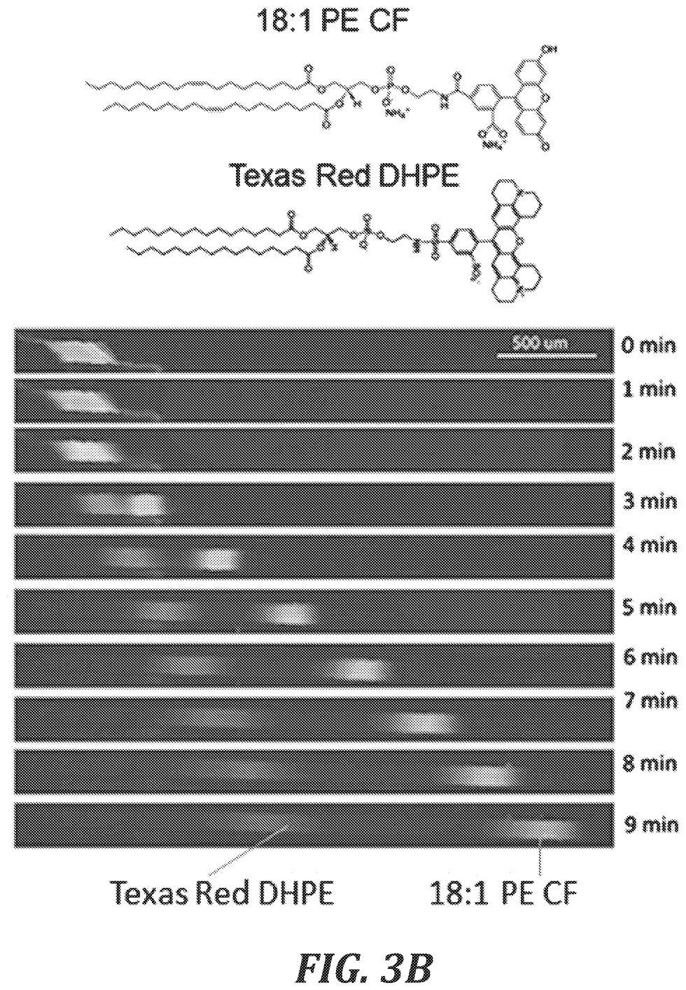

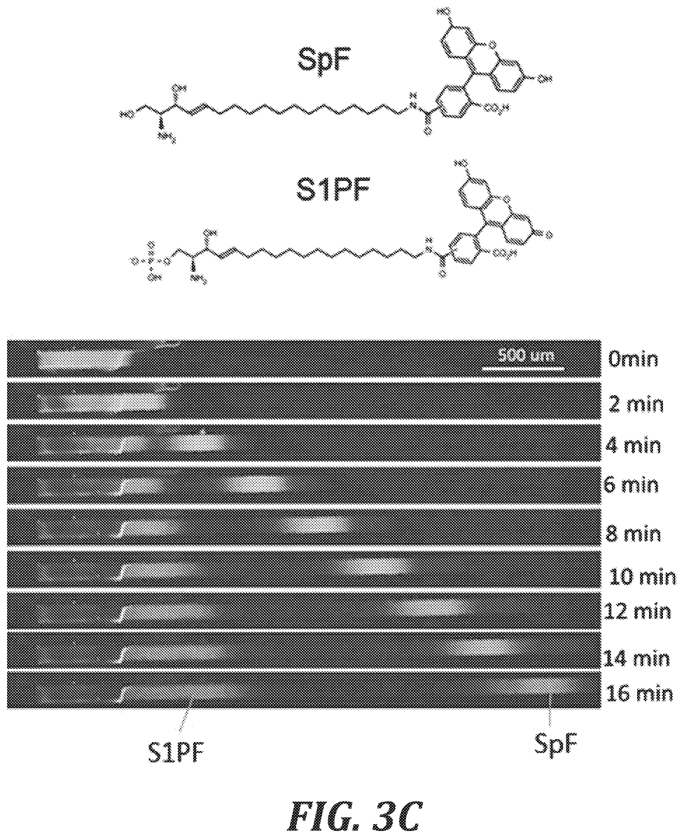

To verify the formation of porous silica monolithic microbands, the glass slide was inspected under a bright field microscope (FIGS. 2B and 2C). The glass channels were filled with silica monolith with their top surface open to air (FIG. 2B). The silica bands are dark and non-transparent due to their porous structure and the difference of refractive index between silica (1.475) and air (1). Because the silica monolith is hydrophilic and water readily enters the porous structure, the bands become clear and transparent upon water immersion (FIG. 3C) due to the similar refractive indices of silica (1.475) and water (1.33).

The above description gives an example of fabrication method (micromolding in 30 microcapillaries or microchannels) to create pTLC bands. However, pTLC can be fabricated from alternative strategies such 3D printing, microtransfer molding, solvent-assisted micromolding, replica micromolding, microcontact molding, photolithography, soft lithography, stereolithography, micro/nano-imprint lithography, electrospinning, and inkjet printing.

### Methods for Using the TLC Devices

In a further aspect, the invention provides methods for using the chromatography device described herein.

In certain embodiments, the invention provides a method for thin layer chromatography of the contents of a single cell, a group of cells, or a micro sample using a chromatography device (i.e., the TLC device) described herein. In certain of these embodiments, the contents are biological analytes. In certain embodiments, the biological analytes are lipids. In other embodiments, the biological analytes are fatty acids, amino acids, peptides, lipopeptides, vitamins, hormones, proteins, carbohydrates, enzyme substrates, and metabolites.

In other embodiments, the invention provides a method for thin layer chromatography of analytes in samples having a volume from about 1 zL to about 100 nL. In certain of these embodiments, the analytes are fatty acids, amino acids, peptides, lipopeptides, proteins, carbohydrates, metabolites, hormones, antigens, pollens, spores, vitamins, cytokines, indoles, antibiotics, pigments, steroids, phenols, bile acids, coumarins, pharmaceuticals, reaction analytes, toxins, pesticides, insecticides, poisons, or pollution contaminants.

In the methods described herein, the sample for chromatography may be deposited by inkjet printing, contact printing, microfluidic delivery, micropipette deposition, spray methods (such as electrospray, electrodeposition), electrowetting, optoelectrowetting, dielectrophoresis, surface acoustic wave deposition, magnetic or electrostatic droplet deposition, immiscible-fluid flows, microtraps, microweirs, microholes, docking sites, microvalving, optical tweezers, or pressure- and magnetic-based manipulation.

In the methods described herein, the sample for chromatography may be migrated by capillary force, pressure, vacuum, centrifugation force, electrophoretic force, osmotic force, or magnetic force.

In the methods described herein, the analytes may be detected by detection known in the art for detecting analytes separate on chromatographic supports. Representative detection techniques include fluorescence absorption (UV, visible, and infrared light), chemical stains, luminescence, infrared spectroscopy, surface-enhanced Raman spectroscopy, mass spectrometry, radioactivity, colorimetry, precipitate formation, electrochemistry, nuclear magnetic resonance, or electrochemiluminescence.

In certain embodiments, the device described herein is used to chromatograph (i) the contents of a single cell, (ii) the contents of a single organism, (iii) the contents of viral particles, or (iv) analytes in samples having a volume from about 1 zL to about 100 nL.

The device described herein may be used to chromatograph samples in forensic drug analysis, toxicology, explosives analysis, and analysis of dyes and inks for the identification and comparison of drugs, explosives, inks and dyes, toxins, and pesticides; and to chromatograph and assay drug candidates and drug metabolites.

The following description demonstrates the separation efficiency representative TLC devices of the invention prepared as described herein.

Separation of spotted lipid mixtures by pTLC. The separation efficiency of a representative pTLC device prepared as described above was evaluated using model lipids. Two model fluorescent lipids (18:1 PE CF and Texas Red DHPE) were selected to determine whether pTLC is capable of separating organic compounds. A parafilm was cut into a sharp triangle shape and its tip was used to micro-spot the sample of 300-1,000 picoliters on the microband of the pTLC device. To spot the sample, the parafilm tip was dipped in fluorescent lipid solution (1 μg/mL in ethanol), taken out, and quickly placed in contact with the sample zone of the pTLC plate. Parafilm is sufficiently soft that it does not damage the silica monolith. After sample spotting, the pTLC device was placed faced down and developed in a petri dish in a horizontal position as shown in FIG. 3A. Solvent was drawn from the wiper at one end into the silica microband. The separation was monitored with an Olympus FluoView FV3000 confocal laser scanning microscope. 18:1 PE CF and Texas Red DHPE was successfully separated using 1-butanol:water:acetic acid (18:4:4 vol:vol:vol) in as little as 3 min with a travel distance as short as 360 μm (FIG. 3B). The average migration speed over 9 min of 18:1 PE CF, 264 μm/min, is more rapid than that of Texas Red DHPE, 101 μm/min. The migration speed is related to the interaction between the compound and silica. Texas Red DHPE has more charged groups (two tertiary amines, one sulfonate) than 18:1 PE CF (one carbonate). Therefore, Texas Red DHPE has more interaction with silica (its surface possesses charges) moving more slowly than 18:1 PE CF.

To further demonstrate the utility of pTLC, two biologically important compounds (sphingosine and sphingosine 1-phosphate) were separated (FIG. 3C). Sphingosine 1-phosphate (S1P) is a metabolic product of sphingosine (Sp) formed by the action of sphingosine kinase (SK) inside cells. Analysis of S1P and Sp of biological samples is useful to reveal lipid signaling in pathology and therapy and is recognized as key mediators in the infection by lipid-enclosed viruses such as SARS, MERS and coronovirus (COVID 19). Due to the cell proliferative properties of S1P, the majority of tumors utilize this pathway to fuel their malignant behavior via genetic mutations, over expression and other strategies to upregulate this pathway. The SK pathway is also involved immune pathologies such as autoimmune diseases. As an example, fingolimod is a sphingosine-1-phosphate receptor modulator used to treat multiple sclerosis. Other morpholino analogues and ozanimod and siponimod are envisioned as novel drug candidates targeting the sphingosine pathway in autoimmune diseases as well as in encephalitis, arthritis, traumatic brain injury, cancer, ulcerative colitis, viral and bacterial infection, fibrosis, renal disease, allograft rejection and other pathologies.

Fluorescein-labeled Sp and S1P solution (10 μM in ethanol) was spotted on a pTLC plate using a parafilm tip. The only difference between sphingosine fluorescein (SpF) and sphingosine 1-phosphate fluorescein (S1PF) is the negative phosphate group in S1PF. SpF was successfully separated from S1PF by developing in 1-butanol:water:acetic acid (18:1:1 vol:vol:vol). The average migration speed over 16 min of SpF, 170 μm/min, is more rapid than that of S1PF, 36 μm/min. The negative phosphate group of S1PF hinders its migration due to its interaction with silica. The above results demonstrate that pTLC is capable of separating micro-spotted organic compounds. One unique advantage of pTLC is that the migration is confined along the microbands without lateral diffusion. Other derivatized lipids (fluorescent or with clickable moieties) are equally suitable for separation and assay by pTLC.

Exemplary lipids that can be analyzed using pTLC are saturated fatty acids, unsaturated fatty acids, phospholipids, sphingolipids, glycolipids, glycerophospholipids, cholesterols, steroid, triacylglycerols, triacylglycerides, fatty acids, bile salts, eicosanoids, ketones, fatty acyls, glycerolipids, saccharolipids, polyketides, glycerophosphocholines, glycerophosphoethanolamines, glycerophosphoserines, glycerophosphoglycerols, glycerophosphates, glycerophosphoinositols, glycerophosphoinositol monophosphates, glycerophosphoinositol bis-phosphates, glycerophosphoinositol tris-phosphates, glycerophosphoglycerophosphoglycerols, cardiolipins, glycerophosphoglycerophosphates, glyceropyrophosphates, CDP-glycerols CDP-DG, glycosylglycerophospholipids, glycerophosphoinositolglycans, glycerophosphonocholines, glycerophosphonoethanolamines.

Other sample deposition strategies with sufficient spatial and volume resolution include inkjet printing, contact printing, microfluidic delivery, micropipette deposition, spray methods (such as electrospray, electrodeposition), electrowetting, optoelectrowetting, dielectrophoresis, surface acoustic wave deposition, magnetic or electrostatic droplet deposition, immiscible-fluid flows, and others. Droplets can be generated by instruments (such as flow cytometry) and deposited directly on pTLC bands. Additionally, microtraps, microweirs, microholes, docking sites, microvalving, optical tweezers or pressure and magnetic-based manipulation can be used for precision sample placement at the sample deposition region.

The above description provides two examples of lipids that can be separated by pTLC. Besides lipids, pTLC is capable to separate other biological analytes including fatty acids, amino acids, peptides, lipopeptides, proteins, carbohydrates, metabolites, hormones, antigens, pollens, spores, vitamins, cytokines, indoles, antibiotics, pigments, steroids, phenols, bile acids, coumarins, pharmaceuticals, reaction analytes, toxins, pesticides, insecticides, poisons, pollution contaminants. In addition to biological analytes, pTLC can be used in forensic drug analysis, toxicology, explosives analysis, and analysis of dyes and inks for the identification and comparison of drugs, explosives, inks and dyes, toxins, pesticides. Of particular value is the assay of drug candidates, drug metabolites and synthetic reaction contaminates or byproducts.

Fluorescence was used as the example for detection of analytes. Other detection strategies are suitable for pTLC (with or without signal amplification) including absorption (UV, visible and infrared light), chemical stains, luminescence, infrared spectroscopy and other spectroscopies, surface-enhanced Raman spectroscopy, mass spectrometry, radioactivity, colorimetry, precipitate formation, electrochemistry, nuclear magnetic resonance, and electrochemiluminescence.

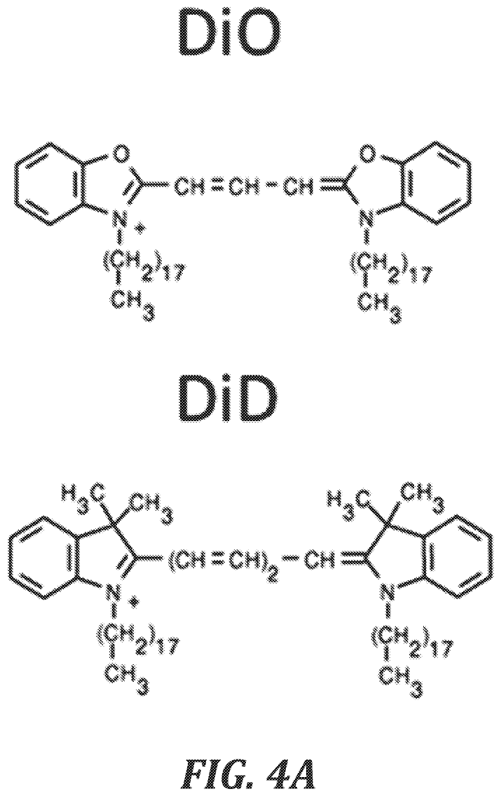

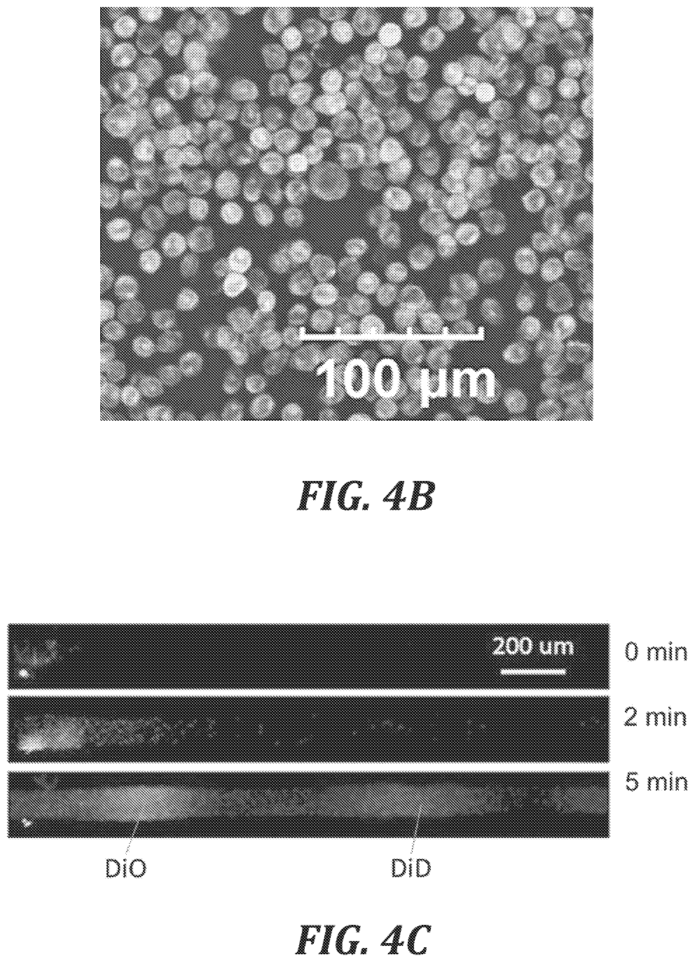

Separation of lipophilic red/green lipids from single cells. To demonstrate the capability of pTLC for single cell analysis, two model fluorescent lipids (DiO and DiD) were first loaded into U937 cells (monocyte-derived tumor cell, volume about 1 pL, and 10-20 μm in size), and then a single U937 cell was spotted on a microband of the representative pTLC device prepared as described above, for separation (FIG. 4). DiO or DiD (chemical structures shown in FIG. 4A) are lipophilic fluorescent stains for labeling cell membranes and other hydrophobic structures. These stains high extinction coefficients and exhibit distinct fluorescence colors (DiO: green fluorescence; DiD: red fluorescence). U937 cells were incubated with DiO and DiD (5 μM in Hanks' balanced salt solution) for 20 min at 37° C. to enable cell uptake of the lipids. The cells were fixed in 4% paraformaldehyde in PBS for 15 min followed by rinsing with PBS for 5 times. Cells exhibited both red and green fluorescence with the expected cell-to-cell heterogeneity (FIG. 4B). To spot a single cell, the parafilm tip was dipped into the cell suspension (10,000 cells/mL), removed, and quickly contacted to the pTLC microband. Due to Poisson statistics, most microbands were spotted with 0 and >1 cells. The number of single cells deposited is readily optimized using microfluidics, inkjet printing, microdroplets, cell docking sites, electrophoresis, contact printing, micropipette deposition, spray methods (such as electrospray, electrodeposition), electrowetting, optoelectrowetting, dielectrophoresis, immiscible-fluid flows, and others. Additionally, microtraps, microweirs, microholes, microvalving, optical tweezers or pressure and magnetic-based manipulation can be used for precision cell placement at the sample deposition region. Only the microbands spotted with 1 cell was used for the assay. The presence of a single cell was confirmed by microscopy. The pTLC plate was developed in 1-pentanol in the setup shown in FIG. 3A. The elution of DiO/DiD from a single cell and migration along the microband was imaged with an Olympus FluoView FV3000 confocal laser scanning microscope (FIG. 4C). DiO and DiD were extracted from the cell and separated in as little as 2 min. In 5 min, the DiD was completely separated from DiO. The migration of DiD is more rapid than that of DiO because DiO is more polar. These results demonstrate that the TLC technology described herein can be applied to assay of the contents of a single cell. The results were accomplished by creating a cell-compatible sample loading zone, microbands that contain the cell contents to minimize dilution, a single-cell deposition strategy, and an adsorbent with cell-size appropriate features. The combination of a sensitive detection method further enhanced the ability to assay the single cell contents.

Besides red and green fluorophores described herein, other fluorophores are suitable for pTLC detection including emission colors ranging from blue, light green, dark green, yellow, orange, red, far red, infrared. These fluorophores span the near-UV, visible and near-IR spectrum.

While the U937 cells are monocyte-derived tumor cells, other tumor and normal cell types can be assayed, for example, lymphocytes, neutrophils, eosinophils, basophils, macrophages, erythrocytes, platelets, megakaryocytes, thrombocytes, mast cells, natural killer cells, plasma cells, epithelial cells, neurons, muscle cells, fat cells, skin cells, nerve cells, endothelial cells, bone cells, fibroblast cells, egg cells, sperm cells, embryonic stem cells, tissue-specific stem cells (e.g., hematopoietic stem cells, mesenchymal stem cells, neural stem cells, epithelial stem cells, skin stem cells), induced pluripotent stem cells, heart cells, intestine cells, muscle, fibroblasts, kidney liver, neuron/nerve, retinal, lung, spleen, bone, and any other cell or tissue found in a mammal, human, plant, insect, or animal.

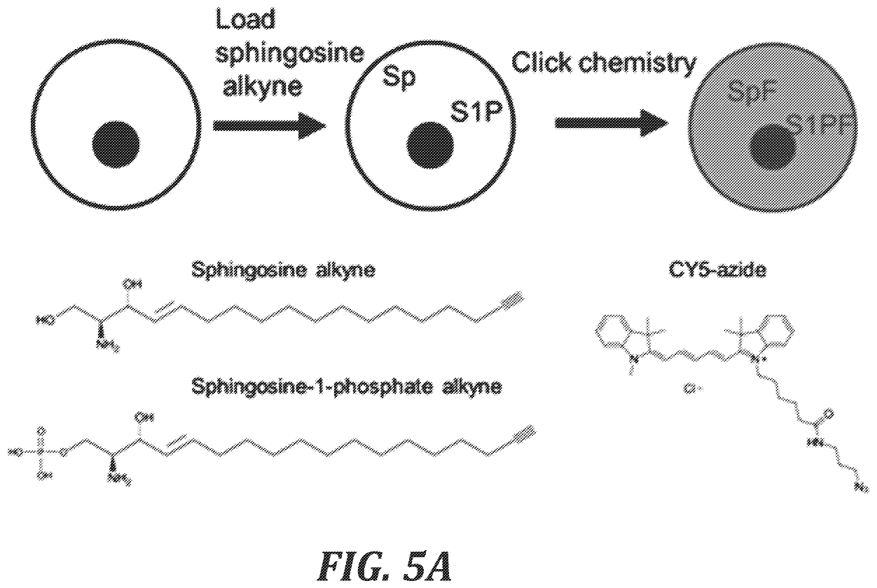

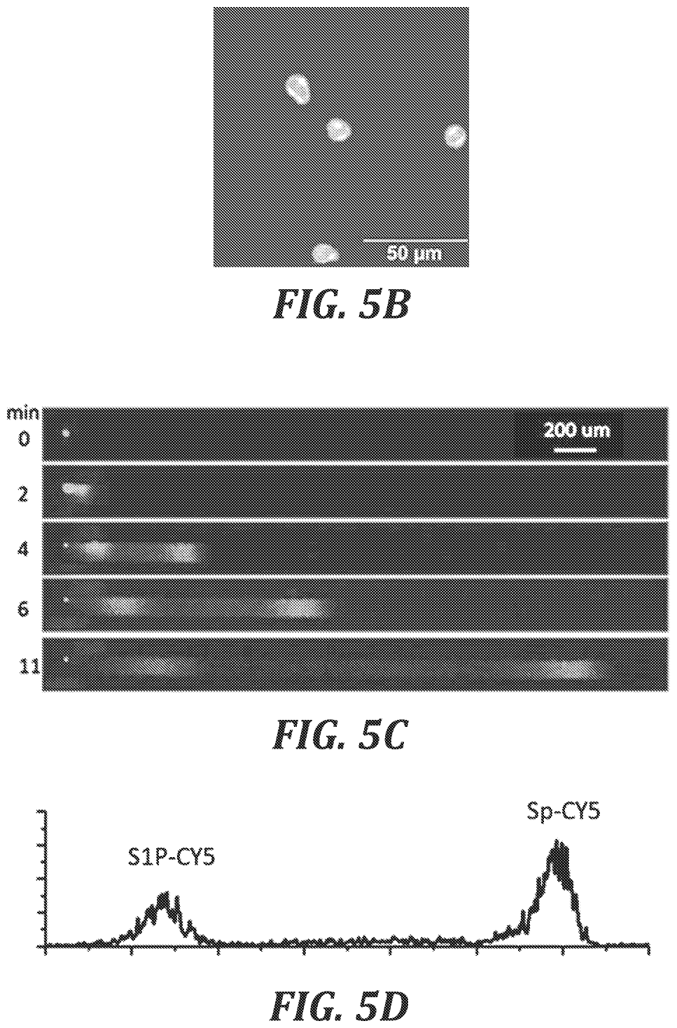

Separation of sphingosine metabolites from single cells. Although DiO or DiD can be separated and detected from a single cell by pTLC as described above, many biologically important compounds inside cells, such as sphingosine and sphingosine 1-phosphate, either are non-fluorescent or are present at a very low concentration. Analyte amplification strategies, highly sensitive detection methods, and sensors can be developed to overcome these challenges. An example of such a strategy follows. Click chemistry was used to conjugate a non-fluorescent sensor loaded inside cells with a highly fluorescent dye for detection purposes (FIG. 5A). U937 cells were incubated with the sensor, sphingosine alkyne (10 μM) for 30 min at 37° C. The sphingosine alkyne sensor enters cells and is metabolized by cellular enzymes much as sphingosine is since the alkyne is minimally perturbative. For example, sphingosine kinase inside the cells converts sphingosine alkyne into sphingosine 1-phosphate alkyne. The ratio of sphingosine alkyne and its metabolite reveals the activity of sphingosine kinase. The cells are incubated with the loaded sphingosine alkyne and then fixed with 4% paraformaldehyde in PBS for 15 min followed by rinsing with PBS for 5 times. To detect the non-fluorescent alkynes, copper (I)-catalyzed azide alkyne cycloaddition (CuAAC), broadly known as click chemistry, was used to conjugate CY5-azide with alkyne moieties inside the cells. CY-5 has a sharp absorption band, high molar extinction coefficients (250,000 cm⁻¹M⁻¹ in ethanol), high quantum efficiency and excellent resistance to photobleaching (FIG. 5B).

To detect the ratio of Sp-CY5 and S1P-CY5 inside a single cell, a single cell was spotted on a pTLC microband of the representative pTLC device prepared as described above, using a parafilm tip. The pTLC plate was developed in 1-butanol:1-propanol:water:triethylamine (18:2:4:1, vol:vol:vol:vol). Sp-CY5 and S1P-CY5 eluted from the single cell and migrated along the microband. CY5 fluorescence was monitored using a microscope (FIG. 5C). Sp-CY5 and S1P-CY5 migrated out and away from the cell within 2 min. The lipids were separated as early as 4 min. After 4 min, the S1P-CY5 was fully separated from Sp-CY5 (resolution=1). The migration of S1P-CY5 is slower than that of Sp-CY5 because S1P-CY5 has an extra phosphate group and so is more polar. Quantitative analysis of the pTLC chromatogram at 11 min shows that the S1P-CY5 peak area is 33.8% and Sp-CY5 peak area is 66.2% of the total CY5 fluorescence measured. From this data, this particular cell is demonstrated to convert 33.8% of the loaded Sp alkyne to S1P alkyne in 30 min via the action of sphingosine kinase. This result demonstrates that the pTLC technology described herein can be used for analyzing biologically important compounds inside single cells.

Examples of other clickable lipids include: PhotoClick sphingosine [(2S,3R,E)-2-amino-13-(3-(pent-4-yn-1-yl)-3H-diazirin-3-yl)tridec-4-ene-1,3-diol], 27-alkyne cholesterol, alkyne-cholesterol, PhotoClick cholesterol, Click PI(4,5)P2-azido, 18:0 propargyl PC, 18:1 propargyl PC, 16:0 propargyl SM (d18:1-16:0), C6(6-azido) Ceramide, C6(6-azido) LacCer, C6(6-azido) GalCer, C6(6-azido) GluCer, 16:0 Azido Coenzyme A, N3C14SOBRAC, Trifunctional Sphingosine, 16:0 azidocaproyl PE, 16:0 hexynoyl PE, 16:0 DBCO PE, 18:1 DBCO PE, 18:0 azidoethyl PC, 16:0 Azidoethyl SM (d18:1/16:0), IKS02, 18:0-16:0(16-azido) PC, DSPE-PEG(2000) azide, DSPE-PEG(2000)-DBCO, trifunctional fatty acid, palmitic acid (15-yne), pacFA, pacFA ceramide, pacFA GlcCer, pacFA GalCer, 16:0-pacFA PC, pacFA-18:1 PC, arachidonic acid-alkyne, oleic acid(17-yne), 16:0(alkyne)-18:1 PC, 16:0(alkyne)-18:1 PE, oleic acid (18-azido), Photoclick Sphingosine-1-Phosphate, Photoclick C6 Ceramide, PhotoClick GM1 (synthetic)- all of which are commercially available including sphingosine alkyne and sphingosine-1-phosphate alkyne. A vast range of other clickable probes suitable for cell-based assays are: other fluorophores, gels, PEG, nucleotides, Bis-dPEG 11-DBCO, Azidopropylvinylsulfonamide Bifunctional Click Reagent, biotin, docosahexaenoic acid alkyne, 4-hydroxy nonenal alkyne, farnesyl alcohol azide, 6-azido hexanoic acid, oleic acid alkyne, eicosapentaenoic acid alkyne, palmitic acid alkyne, 12(S)-HETE-19,20-alkyne, arachidonic acid alkyne, myristic acid alkyne, palmitoyl alkyne-coenzyme A, docosahexaenoic acid alkyne, 4-hydroxy nonenal alkyne, farnesyl alcohol azide, 6-azidohexanoic acid, oleic acid alkyne, L-homopropargylglycine, 4-pentynoyl-coenzyme A, eicosapentaenoic acid alkyne, palmitic acid alkyne, 5-azidopentanoic acid, 12(S)-HETE-19,20-alkyne, arachidonic acid alkyne, myristic acid alkyne, palmitoyl alkyne-coenzyme A (trifluoroacetate salt), 15-hexadecynoyl-CoA, palmitoyl alkyne-CoA, among others.

In certain embodiments, analytes can labeled after separation and prior to detection. In certain embodiments, analytes can be labeled in situ in the sample itself before separation.

In certain embodiments, compounds inside cells can possess both crosslinkable and clickable moieties (e.g., azide, alkyne). In these methods, compounds can be separated by pTLC, crosslinked in the microband to immobilize the compounds, and then clicked with, for example, a fluorescence dye for visualization.

In certain embodiments, the pTLC device of the invention can be used in high-throughput screening for parallel analysis in, for example, drug discovery. Due to the microscale width of each band, a high-density array of microbands can be created. For example, if the width of band is 50 μm, and band gap is 50 μm, 2,000 bands can be created on a 20×20 cm plate. A microdispensor or printer can be used to spot samples on the bands.

In this way, the TLC technology described herein can be advantageously applied to integrated high throughput, and highly parallel microfluidic devices; assays for drug screening; assays for combinatorial chemistry screening; reaction screening; cell-to-cell heterogeneity analyses; subcellular organelle assays; viral detection assays including coronavirus and others such as chickenpox, influenza, herpes, HIV/AIDS, human papillomavirus, mumps, measles, rubella, shingles, hepatitis, meningitis, pneumonia, MERS, SARS, and COVID19. Enveloped viruses are well-suited to these assays.

As set forth above, in certain embodiments, the pTLC device of the invention can be used for very small-scale sample volumes (e.g., 1 zeptoliter to 100 nL) and is thus suitable for analysis of precious, rare or very tiny samples (such as forensic specimens).

As used herein, the term “about” refers to ±5% of the specified value.

While illustrative embodiments have been illustrated and described, it will be appreciated that various changes can be made therein without departing from the spirit and scope of the invention.

1. A thin layer chromatographic (TLC) device, comprising a non-porous substrate having one or more microchannels formed on its surface, each microchannel comprising a porous, exposed, substantially homogeneous microband adapted for fluid flow driven by capillary action.

2. The device of claim 1, wherein the porous microband is a monolithic porous silica microband.

3. The device of claim 1, wherein the fluid flow is capillary fluid flow combined with electrophoresis.

4. The device of claim 1, wherein the fluid flow is capillary fluid flow driven by intermolecular forces.

5. The device of claim 1, wherein the fluid flow is not driven by hydrodynamic centrifugal force or pressure-driven fluid flow.

6. The device of claim 1, wherein the microchannel is in the non-porous substrate surface or formed on top of the non-porous substrate followed by cladding laterally with non-porous walls.

7. The device of claim 1, wherein the microchannel is straight or curved.

8. The device of claim 1, wherein the porous microband has a width from about 1 to about 1000 μm.

9. The device of claim 1, wherein the porous microband has a depth from about 1 to about 100 μm.

10. The device of claim 1, wherein the porous microband has a length substantially co-terminous with the length of the device.

11. The device of claim 1, wherein the porous microband has an aspect ratio (width/depth) from about 1 to about 100.

12. The device of claim 1, wherein the porous microband has a cross-sectional area from about 10 μm² to about 50,000 μm².

13. The device of claim 1, wherein the substrate is glass, quartz, plastic, polymer, ceramic, metal, or silicon.

14. The device of claim 1, wherein the device comprises a single microchannel comprising the porous, substantially homogeneous microband.

15. The device of claim 1, wherein the device comprises from two to about 1,000,000 microchannels, each microchannel comprising the porous, substantially homogeneous microband.

16. A method for fabricating a thin layer chromatography device, comprising;
(a) forming one or more microchannels in a non-porous substrate;
(b) forming a fully enclosed microchannel using a detachable sealing material;
(c) filling the one or more enclosed microchannels with a flowable porous microband precursor composition;
(d) subjecting the non-porous substrate having one or more microchannels filled with a flowable porous microband precursor composition to conditions to that convert the composition to a porous microband; and
(e) detaching the sealing material to expose the porous microbands to provide a thin layer chromatography device.

17-31. (canceled)

32. A method for thin layer chromatography of the contents of a single cell, a group of cells, or a microsample using the device of claim 1.

33-34. (canceled)

35. The method of claim 32, wherein the contents are biological analytes selected from the group consisting of fatty acids, amino acids, peptides, lipopeptides, vitamins, hormones, proteins, carbohydrates, enzyme substrates, and metabolites.

36. A method for thin layer chromatography of analytes in a sample having a volume from about 1 zL to about 100 nL using the device of claim 1.

37. The method of claim 36, wherein the analytes are fatty acids, amino acids, peptides, lipopeptides, proteins, carbohydrates, metabolites, hormones, antigens, pollens, spores, vitamins, cytokines, indoles, antibiotics, pigments, steroids, phenols, bile acids, coumarins, pharmaceuticals, reaction analytes, toxins, pesticides, insecticides, poisons, or pollution contaminants.

38-46. (canceled)
